(19) United States
(12) Patent Application Publication
(10) Pub. No.: US 2023/0385937 A1
(43) Pub. Date: Nov. 30, 2023

(54) REDUCING THE NEED FOR LONG TERM CARE

(71) Applicant: Assured Inc., Wellesley, MA (US)

(72) Inventors: Afik GAL, Needham, MA (US); Roee Nahir, Ramat Hasharon (IL); Yaron David, Haifa (IL); Hila Zadka-Schuldiner, Shoham (IL); Yariv Dror Mizrahi, Ra'anana (IL)

(21) Appl. No.: 18/447,335

(22) Filed: Aug. 10, 2023

Related U.S. Application Data
(63) Continuation of application No. 17/406,142, filed on Aug. 19, 2021.
Application No. 17/406,142 is a continuation-in-part of application No. 17/406,131, filed on Aug. 19, 2021.
(60) Provisional application No. 63/068,062, filed on Aug. 20, 2020.
(60) Provisional application No. 63/068,028, filed on Aug. 20, 2020.

(51) Int. Cl. G06Q 40/06; G06N 7/01; G16H 20/30

(52) U.S. Cl. CPC G06Q 40/06; G06N 7/01; G16H 20/30

(57) ABSTRACT

A method has a model predicting which elderly people are likely to claim for long term care (LTC) within a year based on long term care insurance claims of the elderly people and on assessment data of some of the elderly people. The method includes receiving living environment data of a particular senior with similar characteristics to the elderly people, which includes mobile device patterns of social activity, physical activity, phone use, shopping, sleeping, and waking by the particular senior, from the living environment data, determining a single feature of the features of the model whose change most likely reduces a probability of filing a claim for LTC within a period of time, providing to the particular senior a single intervention related to the single feature, and comparing further patterns of the particular senior with the previous patterns to determine an extent to which the particular senior implemented the intervention.

### CROSS REFERENCE TO RELATED APPLICATIONS

This application is a continuation application of U.S. Ser. No. 17/406,142, filed Aug. 19, 2021, which claims priority from U.S. provisional patent application 63/068,062, filed Aug. 20, 2020, and is also a continuation-in-part application to U.S. Ser. No. 17/406,131, filed Aug. 19, 2021, which claims priority from U.S. provisional patent application 63/068,028, filed Aug. 20, 2020, all of which are incorporated herein by reference.

### FIELD OF THE INVENTION

The present invention relates to insurance generally and to long term care insurance in particular.

### BACKGROUND OF THE INVENTION

Long term care insurance (LTCI) is a relatively new type of insurance that covers the costs of nursing home care and/or long-term care at home. It is typically activated when policy holders become incapacitated in some way but either don't need or don't want to move out of their home in order to receive the care they need.

LTCI insurance is expensive and open-ended, as some of the policy holders will need the care for an extended period of time.

### SUMMARY OF THE PRESENT INVENTION

There is therefore provided, in accordance with a preferred embodiment of the present invention, a model-based method implemented on a computing device having a processor. The method includes having a predictive model built from features based on data of long-term care insurance claims of a block of elderly people at least 80 years old, the block defined by a set of characteristics, and based on assessment data of a least a portion of the elderly people, the predictive model predicting which of the elderly people are likely to claim for long term care within a year. The method includes the processor receiving living environment data for a particular senior between 60 and 80 years old who has the set of characteristics. The living environment data includes patterns generated by a mobile app on a mobile device of the particular senior, the patterns being patterns and irregular patterns of social activity, physical activity, phone use, shopping, sleeping, and waking by the particular senior, the patterns generated from the activity of a plurality of other apps on the mobile device. The method includes the processor determining, using the model on the living environment data, a single feature of the features whose change is most likely to reduce a probability of the particular senior filing a claim for long term care within a predefined period of time, having an intervention table including a plurality of types of interventions, each intervention associating one of the features with at least one intervention, the processor providing to the particular senior a single intervention from the intervention table related to the single feature, and the processor comparing further patterns of the particular senior with the patterns used in the determining to determine an extent to which the particular senior implemented the intervention and if the particular senior achieved an expected health outcome associated with the intervention. The comparing includes checking absolute and relative levels of the activity and their duration, and considering local factors of the particular senior when calculating a rate of change of the activity.

Moreover, in accordance with a preferred embodiment of the present invention, the patterns of social activity include the number of different phone interactions, the frequency and duration of these interactions, and the number of inbound vs outbound interactions.

Further, in accordance with a preferred embodiment of the present invention, the patterns of grocery shopping include identifying large weekly payments, at least two standard deviations above the particular senior's weekly average payment.

Still further, in accordance with a preferred embodiment of the present invention, the patterns of social activity include how often the policy holder goes out of a house, or goes to one of: a malls and a cinema.

Moreover, in accordance with a preferred embodiment of the present invention, the patterns of physical activity include at least one of: driving outside and walking.

Further, in accordance with a preferred embodiment of the present invention, the irregular patterns of physical activity include whether or not the particular senior gets lost when trying to go to known locations.

Still further, in accordance with a preferred embodiment of the present invention, the method includes determining the particular senior's average daily walking hours as a function of cellular phone speed, and noting when the particular senior walks for a significantly longer period of time.

Moreover, in accordance with a preferred embodiment of the present invention, the irregular patterns of sleeping are a function of how many times the particular senior opened the phone during the night.

Further, in accordance with a preferred embodiment of the present invention, the patterns are amount of time spent talking to family members, average daily walking distance, amount of time spent socializing, and number of visits to a recommended social center.

Still further, in accordance with a preferred embodiment of the present invention, the comparing includes determining a percentage of unanswered calls to determine if there was a decrease in hearing.

Finally, in accordance with a preferred embodiment of the present invention, the set of characteristics includes at least one of: an age bracket, a geographic location, and a family situation.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is schematic illustration of a population health management system, constructed and operative in accordance with a preferred embodiment of the present invention;

FIG. 2 is an illustration of an exemplary questionnaire which may be used for an assessment, useful in the method of FIG. 1;

FIG. 3 is an illustration of an exemplary method of scoring the questionnaire of FIG. 2, useful in the method of FIG. 1;

FIG. 4 is a schematic illustration of a method of selecting policy holders to receive different assessments;

FIG. 5 is a schematic illustration of an example selection process to select policy holders for an initial data collection period;

FIG. 6 is a tabular illustration of an intervention table, useful in the system of FIG. 1; and

FIG. 7 is a schematic illustration of an alternative population health management system, constructed and operative in accordance with a second preferred embodiment of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicant has realized that postponing a claim for long term care is good for the policy holders as well as for the insurer. Moreover, Applicant has realized that, by gathering additional data about policy holders (i.e., other than that which insurance companies typically gather about their policy holders), it is possible to find those elderly policy holders who are more likely to activate their long-term care insurance policies in the near term and to provide them with suggestions to achieve a short-term improvement in their health and aging in place status. This may enable those elderly policyholders (i.e., those who are 80 years old and older), to age safely, healthily and independently.

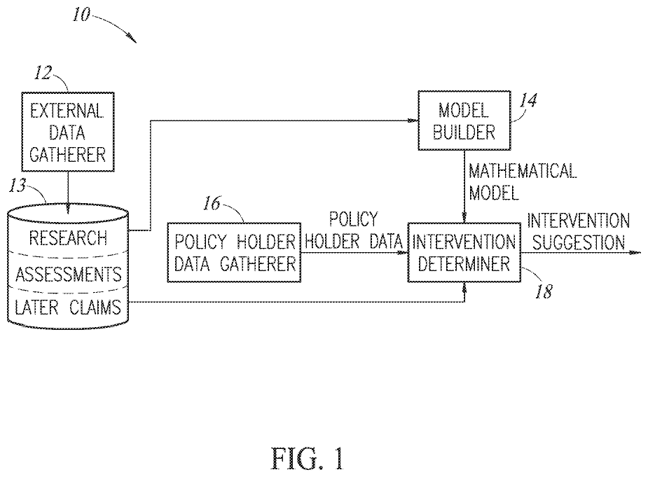

Reference is now made to FIG. 1, which illustrates a health management system 10 for providing intervention suggestions to aid policy holders to not need long term care and/or to remain independent and/or at home, at least for a short term, such as 1 year.

Health management system 10 comprises an external data gatherer 12, a database 13, a model builder 14, a policy holder data gatherer 16 and an intervention determiner 18. External data gatherer 12 may gather data from multiple sources and may store the gathered data in database 13. For example, external data gatherer 12 may receive basic information about policy holders from the insurance company which issued the insurance policies. According to a preferred embodiment of the present invention, external data gatherer 12 may gather assessment data about the policy holders over a period of time, such as 6 months to a year. The assessment data may be the results of questionnaires sent to the policy holders, phone or home assessments made by social workers talking to or visiting the policy holders, observations by medical professionals, etc. At the same time, external data gatherer 12 may receive information about when the policy holders made a claim and for what type of care. External data gatherer 12 may also add research about health issues and, in particular, research about improving quality of life, etc. to the gathered data.

Using at least the assessment data and the claims data, model builder 14 may build a mathematical model, described in more detail hereinbelow, which may predict which candidates are more likely to make claims to long term care and within what time frame. Intervention determiner 18 may use the mathematical model to predict if a particular policy holder may benefit from an intervention, based on the results of recent assessments on that policy holder as gathered by policy holder data gatherer 16.

It will be appreciated that system 10 may provide improved risk management for the insurance company using it, based on improved data collection from policyholders, predictive modeling to target opportunities for intervention and deployment of science-based interventions. System 10 may reduce the likelihood of a claim in the near-term, since some claims will be deferred, while some will be shorter. Moreover, system 10 may monitor the interventions and their results and may update the model as a result.

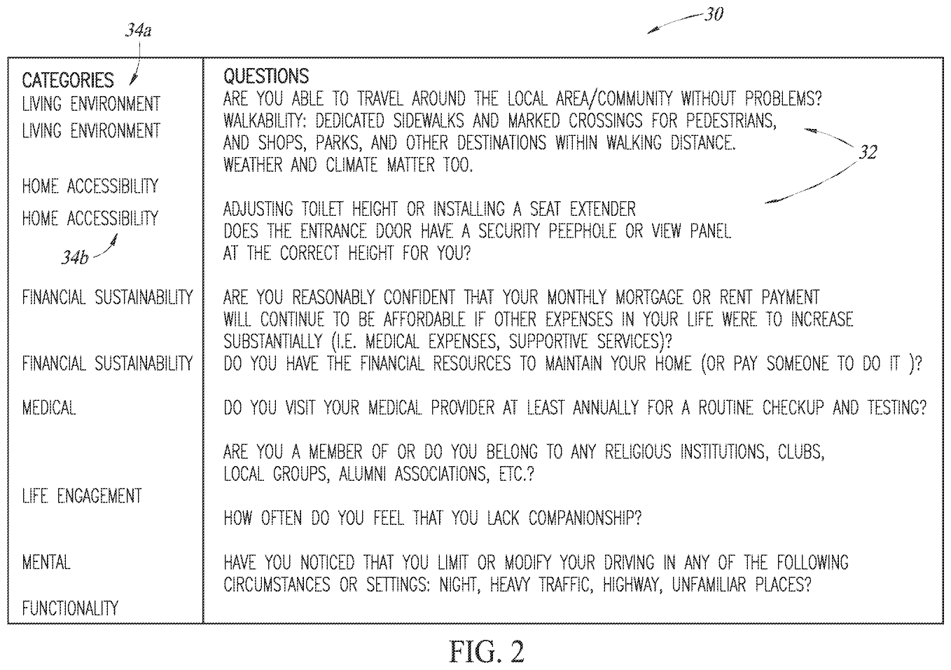

Reference is now made to FIG. 2, which illustrates an exemplary questionnaire 30 which may be used for an assessment, and may be filled in by the policy holder, or by a social worker, nurse or physician, whether during a phone assessment or a home visit.

Each questionnaire 30 may have a plurality of questions 32, which may be categorized into multiple categories 34. For example, category 34a may be “living environment” while category 34b may be “home accessibility”. Category 34a may have questions 32 like “Are you able to travel around the local area/community without problems?” and a question about “walkability” which may ask further questions about the quality of the external environment for walking (i.e., whether or not there are dedicated sidewalks and marked crossings for pedestrians, and shops, parks, and other destinations within walking distance, whether the climate is conducive to regular walking, etc.).

Category 34b may have questions about the feasibility of adjusting certain elements in the premises, about the convenience and presence of security peep holes for the particular policy holder.

FIG. 2 shows additional questions in categories such as financial sustainability, medical issues, life engagement, mental issues and functionality, all of which may affect a person's ability to handle problems when they are arrive, without having to register a claim against their insurance policy.

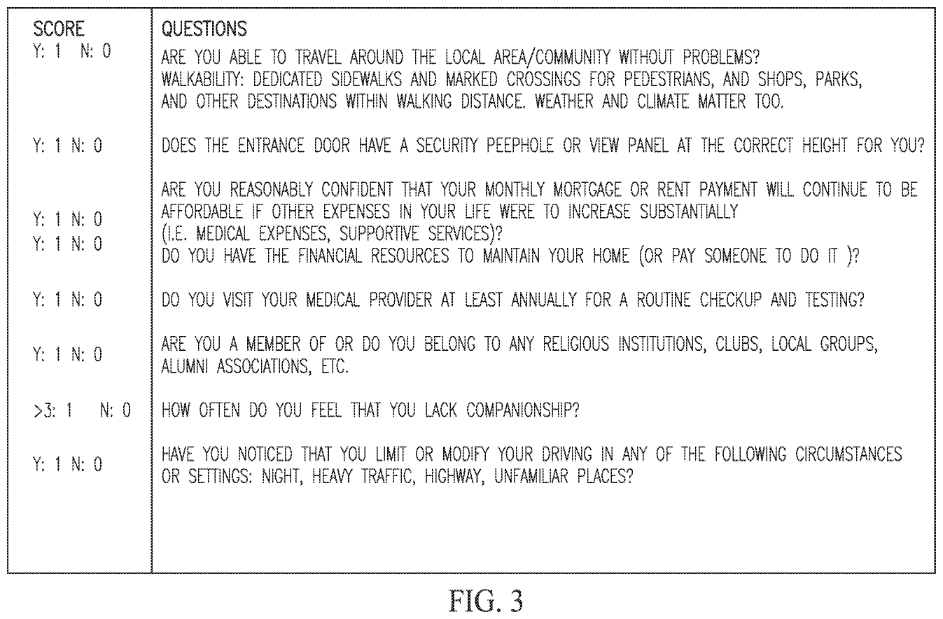

Reference is now made to FIG. 3, which illustrates an exemplary method of scoring questionnaire 30 which may be utilized by external data gatherer 12. Once external data gatherer 12 may finish scoring questionnaire 30, it may store the results in database 13.

As can be seen in FIG. 3, each Yes answer may be given a score of 1 while each No answer may be given a score of 0. Certain questions, like the question “How often do you feel that you lack companionship?” may give a score of 1 if the answer is above a certain number of times. Questions may be positive or negative and scoring may be the same for both or may be different. In the latter, scores to negative questions may be negative. In an alternative embodiment, questions may be kept “in the same direction”. In a further alternative embodiment, questions may be either, so scores of questions that correlate to bad physical/cognitive/mental abilities will increase the global score.

As mentioned hereinabove, external data gatherer 12 may store the scores in database 13, along with any information about the policy holder that it may receive from the insurance company holding the policy. The insurance company typically provides basic contact information, some medical information, and information regarding when and which claims have been made.

External data gatherer 12 may provide assessments to all members of a group, such as a block of policy holders. Alternatively, external data gatherer 12 may reduce the size of the group to be assessed by any suitable method, such as by using a selectable set of different assessments.

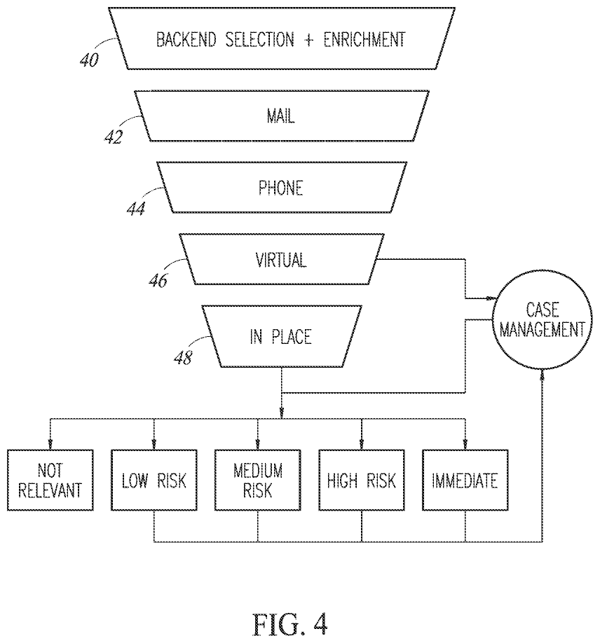

For example, and as shown in FIG. 4 to which reference is now made, external data gatherer 12 may request (step 40) that a user initially define a sub-group of the group, after which external data gatherer 12 may mail (step 42) questionnaires to the members of the sub-group. Of these, external data gatherer 12 may request that a social worker or medical professional call (step 44) some of the “mailed to” members and of those “called to” members, external data gatherer 12 may request that a social worker or medical professional virtually assess (step 46) some of the called members. Finally, external data gatherer 12 may request that a social worker or medical professional may assess in place (step 48) some of the virtually assessed members.

After each type of assessment 40-48, external data gatherer 12 may score the answers according to a pre-defined scoring definition (which may be the scoring method described for FIG. 3 or some other scoring method). External data gatherer 12 may then select those members whose score is above a pre-defined threshold as initial candidates for the next group to be assessed.

To select the next group, external data gatherer 12 may review the current expected costs to assess each of the initial candidates and may select only those candidates whose cost is acceptable. It will be appreciated that candidates that the costs for an in-place assessment may be too high for those that live “far away” from wherever the assessors are. It will also be appreciated that not all the costs are financial. External data gatherer 12 may also include in the cost calculation the risk of upsetting a policyholder, or stimulating a policy holder to ask for services even though it is unlikely that they will receive the intervention, etc. External data gatherer 12 may utilize predetermined cost curves to determine the cost at each level of assessment and these curves may change over time.

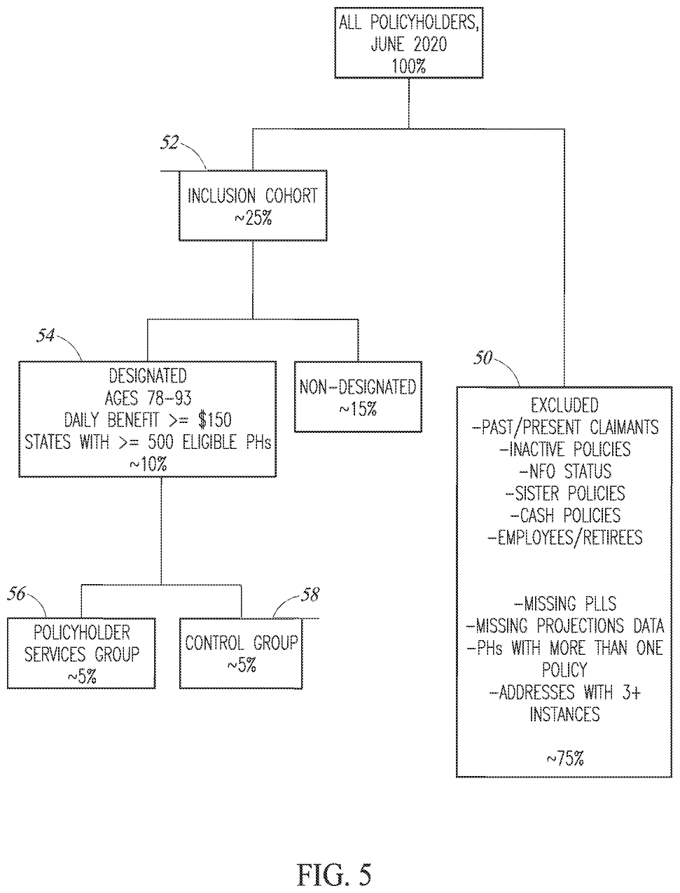

External data gatherer 12 may collect the assessment data described hereinabove as well as claim data (when a claim was made and for what type of care) for a predetermined period of time, such as 6 months or 1 year, to provide sufficient data for model builder 14. FIG. 5, to which reference is now made, illustrates an example selection process to select policy holders for the initial data collection period. The initial selection may remove those policy holders 50 for whom intervention in the short term is unlikely to yield savings, or those who can otherwise not be engaged. The latter include those who have already made claims for long term care, whose policies are inactive, who are employees or retirees of the insurance company, they have more than one policy, more than one address is listed on the policy, etc.

Of the inclusion cohort 52, which in the example of FIG. 5 is 25% of the policy holders, only a portion may meet the intervention criteria, such as they are within the age range for interventions (ages 78-93), they have a daily benefit of over $150, and both policy holders of a joint policy meet the criteria. There may also be criteria related to a cost-benefit analysis determining what region is most beneficial to work in, such as its only cost-beneficial to provide interventions in a state having over 500 eligible policy holders. In FIG. 5, this designated portion 54 is only 10% of the block of policy holders. This may constitute the treatment pool.

For each of the policy holders of the treatment pool, external data gatherer 12 may obtain further information, such as contact information, from the insurance company or, if the insurance company does not have this information, from third party data sources, such as an online address service.

External data gatherer 12 may further define the treatment pool based on who can be contacted, and who among those is willing to engage with the program and may ask those willing to engage to provide information, e.g., by filling out a questionnaire. External data gatherer 12 may assign a score to all those who completed the questionnaire, and may continue the process, as described hereinabove with respect to FIG. 4, at a greater level of detail, for those whose score is above a threshold.

In one embodiment, assessment and claim data may be collected as described above for the treatment pool for the defined period of time. This may provide baseline data relating the assessment data to the claim and may be used to define risk levels (shown in FIG. 4), where those that are expected to file a claim within the next 1-2 years are high risk, within the next 2-3 years are medium risk, and within the next 3-5 years are low risk. Those that are expected to file a claim before the year is out are considered ‘immediate’.

At a later point in time, the treatment pool may be divided in half (as shown in FIG. 5), into a pool 56 to receive interventions and a “control pool” 58 which does not receive interventions. Doing this may enable external data gatherer 12 to associate interventions and the resulting claims, which may enable intervention determiner 18 to generate an intervention table, discussed in more detail hereinbelow, for selecting those scientifically based interventions which may move claims to a later date.

It will be appreciated that the more detailed personal assessments may yield a stratification which may permit an individual treatment plan to be developed for the subset of policyholders whom intervention determiner 18 may determine offer the highest likelihood of a positive economic return relative to the cost of intervening.

Model builder 14 may generate a prediction model from the collected data (assessments and resulting claims) to determine the likelihood (i.e., expected risk level) that each policy holder in the treatment pool will file a claim for long term care within a predefined period of time. Model builder 14 may utilize a predictive model of the type:

$$PC ( age ) = \frac{{e}^{( \sum {α}_{1} pet + {α}_{2} volunteer + {α}_{3} walks + … )}}{1 + {e}^{( {age}^{2} + \sum {α}_{1} pet + {α}_{2} volunteer + {α}_{3} walks + … )}} \qquad ( 1 )$$

where PC(age) is the probability of filing a claim at age X and the features (pet, volunteer, walks, etc.) are the non-medical and medical scores provided through the assessments, most of which are generally not available to insurance companies. For example, some non-medical features might be: things an elderly person does, marital state, financial status, home ownership, social, smoker, etc., while some medical features might be those which can be measured at home, such as blood pressure, temperature, heart rate, etc. As mentioned hereinabove, each response on an assessment is scored and it is this score (1 or 0, per policy holder) which is used to define a value of a feature for model builder 14.

Model builder 14 may train on the data in database 13 to determine the impact coefficients αᵢ for each feature, where the initial values for impact coefficients αᵢ may be determined a priori from research data indicating the importance of one feature or another. During training, model builder 14 may change the values of impact coefficients αᵢ to match the data. It will be appreciated that, after training some impact coefficients αᵢ may be 0 or close to 0, indicating that those features are not likely to affect a claim for long term care.

Model builder 14 may perform a process similar to a logistic regression but one where one input is the age, another input is the square of the age, and some features, such as married and gender, may be co-dependent. Model builder 14 may select features automatically, beginning with age and one other feature and using them to attempt to match the data. Model builder 14 may then pick another feature and may check the extent to which the match to the data has improved. If it has improved, model builder 14 may keep the new feature. Otherwise, it may replace it with a different feature. Model builder 14 may continue the process until it has 4-5 features which, together, may provide the best match to the data in database 13. These features are, then, the ones which are most likely to affect whether or not a policy holder will make a claim at a given age.

In order to check the model, model builder 14 may initially divide the data in database 13 into two or more groups of policy holders and may use one group to determine the model and a second group to check that the model holds for them as well.

Model builder 14 may update the mathematical model over time, as external data gatherer 12 may collect more data and to reflect additional experience and knowledge in data gathering and in use of interventions, discussed in more detail hereinbelow. This may change which features may be included in the model.

Intervention determiner 18 may utilize the mathematical model generated by model builder 14 to define which interventions to suggest for a particular policy holder, once it has received the policy holder's scores from policy holder data gatherer 16.

Specifically, policy holder data gatherer 16 may determine what type of assessment(s) to make on a particular policy holder from among the questionnaire, phone, virtual and in-place assessment options and may score the results. This determination may be according to any suitable decision method, including the cost function method utilized by external data gatherer 12.

Intervention determiner 18 may utilize the mathematical model generated by model builder 14 on the scores received from policy holder data gatherer 16 to determine the risk level of the particular policy holder (i.e., the probability of a claim in the next year (i.e., when the policy holder is 1 year older)). If the risk level of the particular policy holder is high (i.e., above a predetermined level), intervention determiner 18 may rerun the model to see if a change in some feature will reduce the probability of this particular policy holder filing a claim. If so, intervention determiner 18 may suggest one or more interventions related to the changed feature to the particular policy holder. These interventions may be of the type which are designed to increase the likelihood of keeping that policy holder home for another year. Example interventions might be walking every day, some kind of home optimization, such as adding grab bars in the bathroom, engaging in social activities (e.g., religious services, clubs), improving medication administration and support, remote care coordination, managing loss of caregiver, preventing caregiver burnout, providing respite care and educating the policy holder about how to handle his/her diseases.

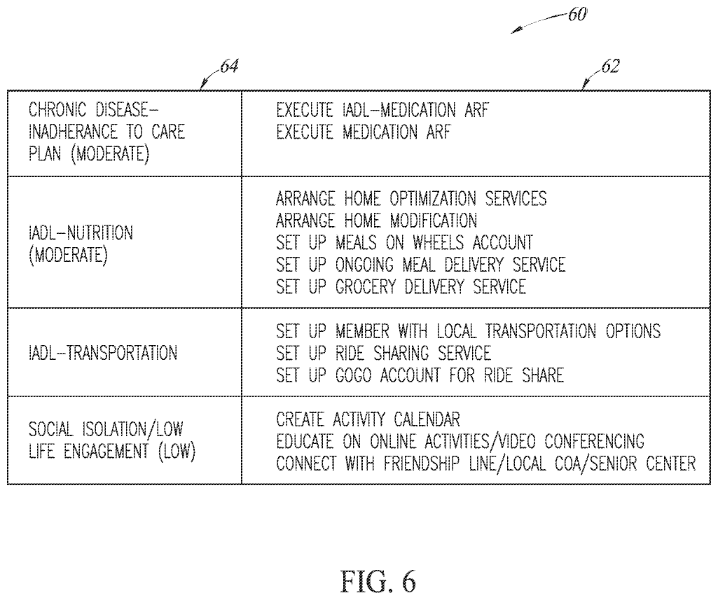

Intervention determiner 18 may utilize an intervention table 60, shown in FIG. 6 to which reference is now made, which may categorize possible interventions 62 according at least to the feature changes 64 determined by intervention determiner 18, since different interventions are known in the scientific literature as being appropriate for different feature changes. For example, if intervention determiner 18 determines that a change in the ‘balance’ feature may affect the probability of a claim from high risk to medium risk, then intervention determiner 18 may suggest a balance intervention, such as arranging for home modification. FIG. 6 shows multiple interventions per changed feature. These various interventions may have an order to them, such that the intervention may begin with the first intervention and, if a later assessment determines it necessary, further interventions may be added.

It will be appreciated that system 10 may be an adaptable system, which may be based on “machine learning”. System 10 may start with an initial model based on research and expert opinions. However, as external data gatherer 12 may gather information from more and more policy holders, model builder 14 may update its models with “field data”. Furthermore, intervention table 60 may be updated as the effectiveness of any intervention may be determined (by research or by the number of claims after it has been used), or with new interventions.

Applicant has realized that the impact coefficients from the mathematical model described hereinabove may be used on similar types of data gathered periodically from younger policy holders (e.g., those who are 60-80 years old) and may be utilized to incentivize the younger policy holders to change their lifestyles. For example, a financial incentive, such as a reduction in life insurance premiums, may reward the adoption of behavioral changes, thereby creating a self-reinforcing beneficial cycle. A system which may provide such an incentive may identify those seniors most likely to adopt behavioral changes and may provide individually tailored solutions for those seniors.

This may be particularly effective because, as Applicant has realized, by the time most people have the inclination or the resources to consider purchasing coverage for long-term care needs, they are too old to purchase such coverage at a reasonable price. By adding such incentives to life insurance policies, their need for long-term coverage may be reduced.

Applicant has realized that, in addition to incentivizing lifestyle changes, such predictions may also be utilized to reduce risk in a life insurance policy and/or to encourage a policy holder to change to a less expensive life insurance policy, etc.

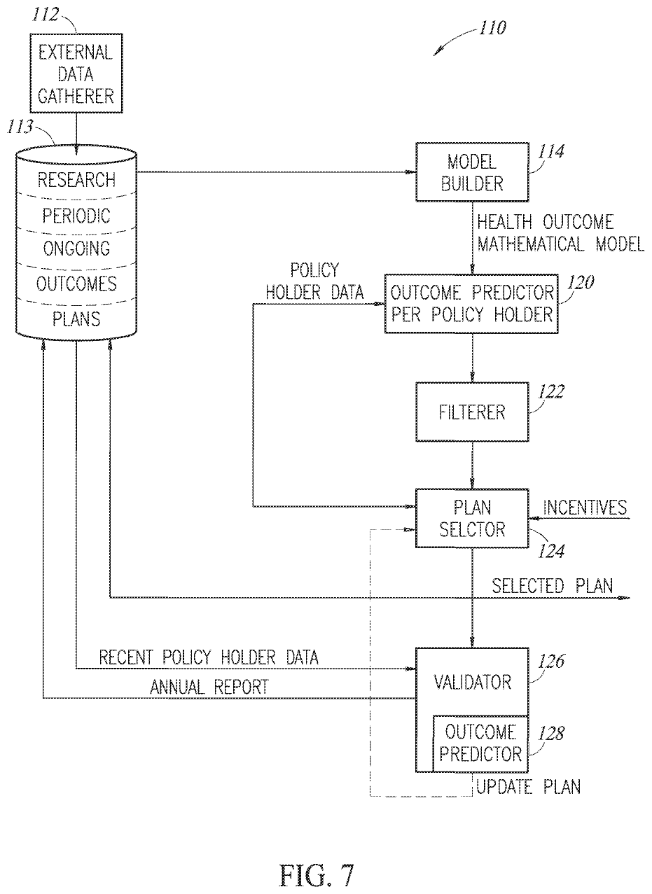

Reference is made to FIG. 7, which illustrates a health management system 110 to provide incentives to life insurance policy holders to improve their lifestyles to attempt to reduce their need for long-term care in the future. As mentioned hereinabove, the life insurance policy holders may be seniors and/or retired persons, generally in the age range of 60-80 years. As will be discussed hereinbelow, these policy holders may be selected from among a plurality of senior policy holders.

System 110 may comprise an external data gatherer 112, a database 113 and a model builder 114, similar to external data gatherer 12, database 13 and model builder 14 of previous system 10. In addition, system 110 may comprise an outcome predictor 120 per policy holder, a filterer 122, a plan selector 124 and a validator 126.

Like external data gatherer 12, external data gatherer 112 may collect data periodically about the plurality of policy holders from multiple sources, including the sources mentioned hereinabove. However, in this embodiment, external data gatherer 112 may additionally collect data from their medical records and from various sensors, such as sensors on smartphones, health trackers, such as those commercially available from FitBit LLC, smart watches, exercise machines and apps, and any other sensors, such as sensors in the home, office and/or fitness premises. The various sensors may provide their data periodically or continually, providing an ongoing source of data about each policy holder.

In one embodiment, external data gatherer 112 may be implemented, at least in part, as a mobile “app” on the policy holder's mobile device (phone, smart watch, tablet, etc.) which may be on or near the policy holder for most hours of the day and may detect the policy holder's activities. For example, it may check social activity, such as the number of different phone interactions, the frequency and duration of these interactions, and the number of inbound vs outbound interactions. It may detect patterns with respect to grocery shopping (such as by identifying large weekly payments, which may be 2 or 3 standard deviations above the policy holder's weekly average payment) and/or how often the policy holder goes out of the house, such as to malls, cinema, etc. It may also detect physical activity, such as driving outside vs. walking. Furthermore, external data gatherer 112 may determine whether or not the policy holder may get lost when trying to go to known locations. The latter may be implemented by determining the policy holder's average daily walking hours (which may be identified from cellular phone speed, etc.) and noting when the policy holder walks for a significantly longer period of time. For example, if the daily average is 45 minutes of walking with a 10 min standard deviation), then external data gatherer 112 may generate a “possible lost” alert if the policy holder walks for 2 or more hours.

As discussed above, the mobile app may detect physical activity. It may also detect sleep and waking patterns. From this, it may determine sleep quality, such as via a function of how many times the policy holder opened the phone during the night.

External data gatherer 112 may store the collected data in database 113, along with ongoing research into quality of life for seniors and with outcomes (claims or otherwise) of lifestyle changes incentivized by system 110.

Model builder 114 may be similar to model builder 14 and may use the impact coefficients αᵢ from model builder 14 in its initial model. However, since model builder 114 may have additional gathered data (such as the data from “wearable” sensors, which may be worn or which may be located near enough to the policy holder, such as smartphones, to detect the policy holder's actions), and since model builder 114 may produce not just risk levels but risk levels per type of health outcome, model builder 114 may generate a health outcome mathematical model. Exemplary health outcomes may be the changed features discussed hereinabove with respect to FIG. 6 which are used to decide on various interventions.

It will be appreciated that model builder 114 may utilize the impact coefficients αᵢ or other functions which may generate health outcomes.

Model builder 114 may also utilize Medicare claims and data from the health and retirement study (HRS), the national long term care survey (NLTCS), the national health and aging trends study (NHATS) and the cardiovascular health study (CHS) datasets to provide further, generalized data.

It will be appreciated that model builder 114 may balance between drivers to aging in place and blockers to aging in place. Drivers to aging in place may be quantified as the sum of the attraction of the physical and/or social environment, perception and emotions (e.g. desire) and actual actions done (e.g. home modification) while blockers to aging in place may be quantified as the sum of unmitigated risks to aging in place, such as, for example, if the policy holder can no longer drive, making it difficult for him/her to shop for needed groceries.

Model builder 114 may generate a claim prediction, which may be the likelihood of needing long term care and may be a function of a policy holder's disability, his/her rate of deterioration in physiological reserve, and his/her need for medium to large amounts of help vs his/her desire to age in place independently.

Outcome predictor 120 may use the health outcome mathematical model produced by model builder 114 on the gathered data of a set of policy holders of a given age (e.g., 60-62, 62-64, etc. to generate probabilities of health outcomes for each policy holder of that age and may provide the per-policy-holder health outcomes to filterer 122.

Filterer 122 may review the probabilities of the health outcomes for the set of policy holders and may select those health outcomes whose probabilities are higher than pre-determined thresholds to be risk factors for that age group.

Plan selector 124 may consider each policy holder separately and may review the policy holder's gathered data along with the determined risk factors from filterer 122 and the incentive options currently available from the insurance company. These incentive options may be cash, a reduction in the cost of a life insurance policy, an intervention, etc., and may be ones that have proved successful for others of the particular senior's age bracket. Plan selector 124 may utilize this information to generate a health improvement plan for that particular senior policy holder and may store the selection in an incentivation record of database 113.

Validator 126 may periodically (such as annually) check the data for each policy holder which received an incentive to see if the expected outcome for that particular policy holder was achieved. Validator 126 may look at the plan and at the recent policy holder data gathered by external data gatherer 112 and may determine the extent to which the policy holder implemented the recommended action(s) and/or the extent to which the recommended action(s) had the predicted result. Validator 126 may update the incentivation record accordingly.

Validator 126 may compare the patterns of current actions (e.g., amount of time spent talking to family members, average daily walking distance, amount of time spent socializing, number of visits to a recommended social center, etc.) of the policy holder determined from an annual reassessment and from the ongoing and periodic data from the mobile app and other sensors, with patterns of such actions before the policy holder received the health improvement plan and/or with a defined “normal” for other people in the policy holder's demographic, such as age bracket, geographic location, family situation, etc.

Validator 126 may check activity types and duration, both their absolute levels (e.g., speed, duration, distance) and their relative levels (is the policy holder walking more or less than before, faster or slower, etc.), what is the rate of change (e.g., shallow decline or steeper decline), etc. To determine the rate of change, validator 126 may also factor in weather and other local factors, which may include a comparison to other people in the same geography.

Validator 126 may determine if there was a decrease in mobility and/or in hearing (where the latter may be determined at least from a percentage of unanswered calls). Validator 126 may utilize the pulse rate and heart activity information from a smart watch, health tracker, or smartphone, and may also access smart medical records, if there are any.

In addition, validator 126 may include its own outcome predictor 128 to which it may provide the events it has deduced in the previous year of data. Outcome predictor 128 may utilize the same health outcome mathematical model to determine a new set of health outcomes, their probabilities and risks, which may indicate the updated trajectory of aging, and may compare the updated health outcomes to those of the previous year(s).

Validator 126 may provide the results to plan selector 124 to update the incentivization plan, where progress vs. goals from the previous year is evaluated and “points/coverage dollars” are given out of an annual maximum (some are given for participation in the assigned program and some for positive changes in the scores).

Plan selector 124 may then generate an updated plan, which may include an option to increase coverage in a way that keeps the level of risk the same or lower than it was previously. For example, the policy holder may be given an option to purchase additional long-term coverage at the same initial guaranteed price. This additional coverage may be capped, such as at $50-100K, and the policy holder may be given a guarantee that the coverage cannot decrease unless there is something fraudulent in their results.

It will be appreciated that system 110 may enable life insurance companies to replace at least some of the underwriting of such policies with the deferring of long-term care benefits.

Moreover, system 110 may provide a multi-layered approach to reducing disability and its cost for policy holders who are senior citizens (60+ years of age). The goal of system 110 may be to prevent problems before they occur and it may attempt to enable healthy and financially sustainable aging in place. The goals may include prevention of diseases, improvement in home safety and function, establishment and support of a caregiving circle for the senior citizen, prevention of further deterioration, and minimization of the cost of care.

Unless specifically stated otherwise, as apparent from the preceding discussions, it is appreciated that, throughout the specification, discussions utilizing terms such as “processing,” “computing,” “calculating,” “determining,” or the like, refer to the action and/or processes of a general purpose computer of any type, such as a client/server system, mobile computing devices, smart appliances, cloud computing units or similar electronic computing devices that manipulate and/or transform data within the computing system's registers and/or memories into other data within the computing system's memories, registers or other such information storage, transmission or display devices.

Embodiments of the present invention may include apparatus for performing the operations herein. This apparatus may be specially constructed for the desired purposes, or it may comprise a computing device or system typically having at least one processor and at least one memory, selectively activated or reconfigured by a computer program stored in the computer. The resultant apparatus when instructed by software may turn the general-purpose computer into inventive elements as discussed herein. The instructions may define the inventive device in operation with the computer platform for which it is desired. Such a computer program may be stored in a computer readable storage medium, such as, but not limited to, any type of disk, including optical disks, magnetic-optical disks, read-only memories (ROMs), volatile and non-volatile memories, random access memories (RAMs), electrically programmable read-only memories (EPROMs), electrically erasable and programmable read only memories (EEPROMs), magnetic or optical cards, Flash memory, disk-on-key or any other type of media suitable for storing electronic instructions and capable of being coupled to a computer system bus. The computer readable storage medium may also be implemented in cloud storage.

Some general-purpose computers may comprise at least one communication element to enable communication with a data network and/or a mobile communications network.

The processes and displays presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the invention as described herein.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A model-based method, the method implemented on a computing device having a processor, the method comprising:
having a predictive model built from features based on data of long-term care insurance claims of a block of elderly people at least 80 years old, said block defined by a set of characteristics, and based on assessment data of a least a portion of said elderly people, said predictive model predicting which of said elderly people are likely to claim for long term care within a year;
said processor receiving living environment data for a particular senior between 60 and 80 years old who has said set of characteristics, said living environment data comprising patterns generated by a mobile app on a mobile device of said particular senior, said patterns being patterns and irregular patterns of social activity, physical activity, phone use, shopping, sleeping, and waking by said particular senior, said patterns generated from the activity of a plurality of other apps on said mobile device;
using said model on said living environment data, said processor determining a single feature of said features whose change is most likely to reduce a probability of said particular senior filing a claim for long term care within a predefined period of time;
having an intervention table comprising a plurality of types of interventions, each intervention associating one of said features with at least one intervention;
said processor providing to said particular senior a single intervention from said intervention table related to said single feature; and
said processor comparing further said patterns of said particular senior with said patterns used in said determining to determine an extent to which said particular senior implemented said intervention and if said particular senior achieved an expected health outcome associated with said intervention; wherein said comparing comprises:
checking absolute and relative levels of said activity and their duration; and
considering local factors of said particular senior when calculating a rate of change of said activity.

2. The method of claim 1 wherein said patterns of social activity comprise the number of different phone interactions, the frequency and duration of these interactions, and the number of inbound vs outbound interactions.

3. The method of claim 1 wherein said patterns of grocery shopping comprise identifying large weekly payments, at least two standard deviations above the particular senior's weekly average payment.

4. The method of claim 1 wherein said patterns of social activity comprise how often the policy holder goes out of a house, or goes to one of: a malls and a cinema.

5. The method of claim 1 wherein said patterns of physical activity comprise at least one of: driving outside and walking.

6. The method of claim 1 wherein said irregular patterns of physical activity comprise whether or not the particular senior gets lost when trying to go to known locations.

7. The method of claim 6 and comprising determining the particular senior's average daily walking hours as a function of cellular phone speed, and noting when the particular senior walks for a significantly longer period of time.

8. The method of claim 1 wherein said irregular patterns of sleeping are a function of how many times the particular senior opened the phone during the night.

9. The method of claim 1 wherein said patterns are amount of time spent talking to family members, average daily walking distance, amount of time spent socializing, and number of visits to a recommended social center.

10. The method of claim 1 wherein said comparing comprises determining a percentage of unanswered calls to determine if there was a decrease in hearing.

11. The method of claim 1 wherein said set of characteristics comprises at least one of: an age bracket, a geographic location, and a family situation.
